(19) United States
(12) Patent Application Publication
(10) Pub. No.: US 2025/0235491 A1
(43) Pub. Date: Jul. 24, 2025

(54) METHOD FOR ENHANCING IMMUNE SYSTEM FUNCTIONS IN A SUBJECT

(71) Applicant: Macularity, Inc., Poway, CA (US)

(72) Inventor: Connor McAulay, Poway, CA (US)

(73) Assignee: Macularity, Inc, Poway, CA (US)

(21) Appl. No.: 18/985,909

(22) Filed: Dec. 18, 2024

Related U.S. Application Data
(63) Continuation of application No. 18/368,888, filed on Sep. 15, 2023.
(60) Provisional application No. 63/405,101, filed on Sep. 9, 2022.

(51) Int. Cl. A61K 35/748; A61K 31/336; A61K 36/05; A61K 36/45

(52) U.S. Cl. CPC A61K 35/748; A61K 31/336; A61K 36/05; A61K 36/45

(57) ABSTRACT

The present application discloses a method for enhancement of immune system functions in a subject. The method involves providing the subject with a spirulina or a spirulina-based formulation obtained from Arthrospira genus and/or Limnospira genus. This administration would merely depend upon the dosage amount and administration schedule thereof to the subject. For example, dosage amount, gap between doses, etc.

### CROSS REFERENCE TO RELATED APPLICATION

This application is a Continuation-In-Part, and claim the benefit of, U.S. application Ser. No. 18/368,888, filed on Sep. 15, 2023, and entitled METHOD FOR ENHANCING IMMUNE SYSTEM FUNCTIONS IN A SUBJECT; which further claims the benefit of U.S. provisional application Ser. No. 63/405,101 filed on Sep. 9, 2022. All of the above-captioned disclosures are incorporated by reference herein in their entirety.

### TECHNICAL FIELD

The present application relates to a method for enhancing immune system functions in a subject. The present application also discloses a pre-defined administration schedule for administering a spirulina or a spirulina-based formulation for enhancing immune system functions and for the treatment of diseases of the eye, including age-related macular degeneration in a subject. The present application also discloses a method for reversing age-related macular degeneration in a subject.

### BACKGROUND

Efficiency of any drug to reach a particular target and treat a disorder or a disease depends upon a number of factors. For example, constituents present in the drug, size of the drug, mode of delivery thereof, biodegradability and other physicochemical parameters of the drug molecule. Apart from such physio-chemical and biological parameters, there are other parameters also equally important, for example, dosage amount, and frequency of intake of the drug. There are many drugs available to treat the same particular disease, however, efficiency thereof may vary. There are many drugs which have constituents/extracts obtained from a variety of genus species. For example, Spirulina is a well-known blue-green alga that is quite acknowledged for various benefits thereof. It has been well used for its nutritional and anti-inflammatory properties for upwards of 500 years. It has been also well acknowledged for its constituents—proteins, and vitamins in higher amounts, making it a suitable dietary supplement for people on vegetarian or vegan diets. It is widely used as a nutrient supplement for its anti-inflammatory and nutritional properties. It helps to regulate the immune system and possesses antioxidant and inflammation-fighting properties.

There have been many formulations to regulate the immune system. To further elevate the immune system functions in a subject, many experiments have been conducted on animals. In one example, zebrafish are used as a subject to show the efficiency of spirulina to elevate immune response. However, further efficiency of the spirulina to treat or reverse or cure certain ailments largely depends upon the dosage amount and administration schedule of the spirulina-based formulation. There are many such products available in the market. For example, a cream formulation contains Spirulina Platensis powder with different Nonionic Surfactants for the treatment of acne vulgaris. In some compositions, high replacement hyprolose is added to spirulina. Some manufacturers add coatings to existing formulations. There are many methods for the preparation of such formulations available in conventional methods. However, all thereof emphasize the nutritional content of such formulations which tend to enhance immune system functions in a subject. However, the efficiency of the formulations to further enhances the immune system in such a capacity that it can reverse the disease.

Therefore, there exists a need for improvising the dosage and administration schedule of existing formulation to be administered to a subject so that there can be a further enhancement in immune response in the subject.

### SUMMARY

In a first aspect, the present application discloses a method for enhancing immune system functions in a subject. The method comprises providing a predefined administration schedule administering a spirulina or a spirulina-based formulation in a unit dosage form effective to enhance the immune system. The formulation may include 86% spirulina, cyanobacteria, 5% of chlorella, 7% of bilberry extract, and 2% of fucoxanthin. The spirulina is selected from one or more of species consisting of Limnospira fusiformis, Limnospira indica, Limnospira maxima, Arthrospira platensis, Arthrospira erdosensis, Arthrospira jenneri, Arthrospira fusiformis, Arthrospira indica, and Arthrospira maxima.

In an embodiment, the predefined administration schedule comprises a number of dosages of the formulation and gap between the dosages along with the dosage amount. The method includes providing the spirulina or the spirulina-based formulation in a unit dosage form in the range of 2000 mg-4000 mg per day.

In another embodiment, the method involves administering a number of dosages of the spirulina or the spirulina-based formulation in the range of 4-6 doses per day with a gap between two doses in a time range of 90-240 minutes. The method involves enhancing healing and reducing inflammation in the subject.

As a second aspect, the present application discloses the use of the spirulina or the spirulina-based formulation for enhancing immune system functions in a subject. The formulation is administered to the subject in a unit dosage form in the range of 2000 mg-4000 mg per day and number of dosages thereof in the range of 4-6 doses per day with a gap of time in the range of 90-240 minutes between two dosages.

As a third aspect, the present application discloses a method for reversing age-related macular degeneration in a subject. The method includes providing a predefined administration schedule for administering a spirulina or a spirulina-based formulation in a unit dosage form effective to reverse the age-related macular degeneration in the subject.

In an embodiment, unit dosage amount of the spirulina or the spirulina-based formulation in a unit dosage form in the range of 2000 mg-4000 mg per day.

In another embodiment, the spirulina or the spirulina-based formulation may be administered in the range of 4-6 doses per day with a gap of time in the range of 90-240 minutes between two dosages.

As a fourth aspect, the present application discloses use of the formulation for the treatment of age-related macular degeneration in the subject. The formulation is administered to the subject in a unit dosage form in the range of 2000 mg-4000 mg per day. Number of dosages of the formulation in the range of 4-6 doses per day and gap between two doses in a time range of 90-240 minutes.

As a fifth aspect, the present application discloses a method for curing eye-related ailments in a subject. The method includes providing a predefined administration schedule for administrating a spirulina or a spirulina-based formulation in a unit dosage form effective to cure the eye-related ailments.

In an embodiment, the method includes providing the formulation in a unit dosage form in the range of 2000 mg-4000 mg per day. The method includes administering a number of dosages of the formulation in the range of 4-6 doses per day with a gap of time in the range of 90-240 minutes between two dosages.

As a seventh aspect, the present application discloses use of the formulation for curing eye-related ailments.

### BRIEF DESCRIPTION OF DRAWINGS

The accompanying FIGURES (FIGS.) illustrate embodiments and serve to explain the principles of the disclosed embodiments. It is to be understood, however, that these FIGURES are presented for purposes of illustration only, and not for defining the limits of relevant applications.

FIG. 1 shows a table depicting dosage and administration schedule of a spirulina or a spirulina-based formulation for the treatment of various ailments.

### DETAILED DESCRIPTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

The present application pertains to a method for enhancing immune system functions in a subject. The subject is administered with a spirulina or a spirulina-based formulation to treat a number of diseases including such as but not limited to enhancing the immune system, eye ailments, and so on. The formulation may include 86% spirulina, cyanobacteria, 5% of chlorella, 7% of bilberry extract, and 2% of fucoxanthin. The spirulina is selected from one or more of species consisting of Limnospira fusiformis, Limnospira indica, Limnospira maxima, Arthrospira platensis, Arthrospira erdosensis, Arthrospira jenneri, Arthrospira fusiformis, Arthrospira indica, and Arthrospira maxima. This formulation is efficient enough to enhance immune system functions in the subject. However, efficiency thereof may be further elevated. Such an elevation depends upon a number of parameters. The parameters include, but are not limited to, dosage amount, number of doses, the gap between the doses, and so on.

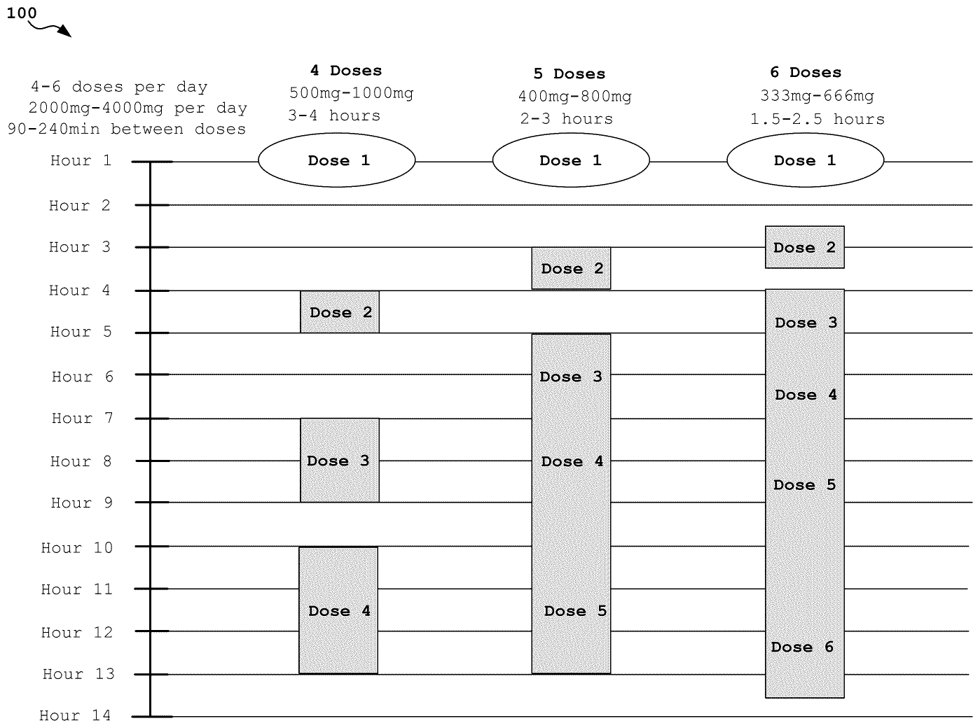

FIG. 1 shows table 100 depicting different embodiments of the dosage amounts of the formulation and administration schedule corresponding to the number of doses. The number of doses depends upon severity of disease and age of the subject. For example, if the subject is a child, the subject may require less dosage amount and lesser number of dosages. In another embodiment, if the subject is an old person and is very ill or diseased with diseases such as macular degeneration, then in such a case, frequent doses per day may be required for the subject.

As shown in FIG. 1, maximum dosage amount may be in the range of 2000-4000 mg per day along with 4-6 doses per day. In an embodiment, there may be a gap of 90-240 minutes between the two doses. The gap between any two doses may be of predetermined equal intervals. In some embodiments, the gap between any two doses may be more than that of the gap between any other doses in a day. In some embodiments, the gap between any two doses may be of predetermined unequal intervals. In some embodiments, one dose may be administered in every fixed span of hour. In some embodiments, number of the dosage amounts in a day may vary in quantity. In some embodiments, number of dosages for particular number of days or weeks may vary. In some embodiments, number of dosages amounts in quantity and number may vary on alternative days. As the subject shows positive changes in the treatment, the dosage amounts of the formulation may be reduced.

If there are 4 number of doses to be taken up, then each dose may be in the range of 500-1000 mg per day. There may be a gap of 3-4 hours between every two doses. For example, there may be four hours gap between Dose 1 and Dose 2. However, there may be one hour gap between dose 2 and dose 3 while only one-hour gap between Dose 3 and dose 4. There may be an embodiment in which the dosage amount to be administered is not fixed to be taken at a particular time. For example, Dose 4 can be taken within 10 to 13 hours and Dose 3 can be taken within 7-9 hours.

In another exemplary embodiment, there may be 5 doses to be administered. The dosage amount may vary in the range of 400-800 mg and the gap between the doses may be in the range of 2-3 hours. There may be a gap of two hours between Dose 1 and Dose 2. There may be one hour gap between Dose 2 and Dose 3. Then, in any of next three hours, the dose 4 may be administered. Furthermore, Dose 5 may be administered at intervals of any next 3 hours.

In another example of 6 doses per day, the dosage amount may vary in the range between 333-666 mg with a gap between 1.5 and 2.5 hours. There may be a 2-3 hours gap between Dose 1 and Dose 2. There may be half-hour gap between Dose 2 and Dose 2. Following therefrom, Doses 4, 5, and 6 may be administered any time in the next span of 6-13 hours. There may be a fixed sequence to be followed in the number of doses. In some embodiments, the sequence of the number of doses may be unfixed. The dosage amount also in each dose in a day may vary from each other. In some embodiments, the dosage amount in each dose in a day may be of same amount.

The aforementioned administration schedule may be efficient to enhance the immune functions of the subject. For example, it may be used in curing eye related ailments, and usage in temporarily enhancing the immune system's functions without triggering an autoimmune response, respectively.

The aforementioned exemplary schedules aim to treat or lessen the severity of immune deficiencies and related disorders, and/or the symptoms associated with said disorders. For example, age-related macular degeneration, a condition resulting from an immune deficiency, may be one such disease. This condition can be reversed for the first time through the use of such schedule. The schedule can enhance efficiency of the formulation by “enhancing” the immune system, speeding up and enhancing many processes without inducing an autoimmune response capable of harming the user's cells. This may be able to treat and reverse macular degeneration and is likely applicable to many other disorders considering its potential to enhance healing processes and reduce inflammation significantly, among other benefits.

In the application, unless specified otherwise, the terms “comprising”, “comprise”, and grammatical variants thereof, are intended to represent “open” or “inclusive” language such that they include recited elements but also permit the inclusion of additional, non-explicitly recited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. The description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Numerous embodiments of the invention are possible. The previous exemplary embodiments are intended to merely illustrate, and not limit, the breadth and depth of embodiments that can fall within the scope of the appended claims and future claims, which define the invention. For example, the apparatus will be scaled to accommodate different flow rates of the water to be treated or impregnated. The chemical flow rates, hence, the concentration of the chemistry, and the pressure in the system may be adjusted depending on the contaminants to be treated and/or the particular application.

It will be apparent that various other modifications and adaptations of the application will be apparent to the person skilled in the art after reading the foregoing disclosure without departing from the spirit and scope of the application and it is intended that all such modifications and adaptations come within the scope of the appended claims.

What is claimed is:

1. A method for enhancing immune system functions in a subject, the method comprising providing a predefined administration schedule administering a spirulina or a spirulina-based formulation in a unit dosage form effective to enhance the immune system.

2. The method of claim 1, wherein the predefined administration schedule comprising number of dosages of the formulation and gap between the dosages along with the dosage amount.

3. The method of claim 1, wherein the formulation comprising 86% spirulina, a type of blue-green algae, 5% chlorella, 7% bilberry extract, and 2% fucoxanthin.

4. The method of claim 1, comprising providing the formulation in a unit dosage form in the range of 2000 mg-4000 mg per day.

5. The method of claim 1, comprising administering number of dosages of the formulation in the range of 4-6 doses per day with a gap between two doses in a time range of 90-240 minutes.

6. The method of claim 1, comprising enhancing healing and reducing inflammation in the subject.

7. The method of claim 1, wherein the spirulina is selected from one or more of species consisting of Limnospira fusiformis, Limnospira indica, Limnospira maxima, Arthrospira platensis, Arthrospira erdosensis, Arthrospira jenneri, Arthrospira fusiformis, Arthrospira indica, and Arthrospira maxima alone or combinations thereof.

8. The method of claim 2, wherein the gap between two doses is of predetermined equal intervals.

9. The method of claim 2, wherein the gap between any two doses, is more than that of the gap between any other doses in a day.

10. The method of claim 2, wherein the gap between any two doses is of predetermined unequal intervals, and wherein one dose is administered in every fixed span of hour.

11. The method of claim 2, wherein number of dosages in a day/week varies as compared to that of the next day/week.

12. A method for reversing age-related macular degeneration in a subject, the method comprising providing a predefined administration schedule for administering a spirulina or a spirulina-based formulation in a unit dosage form effective to reverse the age-related macular degeneration in the subject.

13. The method of claim 12, wherein the formulation is administered to the subject in a unit dosage form in the range of 2000 mg-4000 mg per day and number of dosages thereof in the range of 4-6 doses per day with a gap of time in the range of 90-240 minutes between two dosages.

14. The method of claim 12, comprising providing the formulation in a unit dosage form in the range of 2000 mg-4000 mg per day.

15. The method of claim 12, comprising administering a number of dosages of the formulation in the range of 4-6 doses per day with a gap of time in the range of 90-240 minutes between two dosages.

16. A method for curing eye-related ailments, the method comprising providing a predefined administration schedule for administering a spirulina or a spirulina-based formulation in a unit dosage form effective to cure eye related ailments.

17. The method of claim 16, wherein the formulation is administered to the subject in a unit dosage form in the range of 2000 mg-4000 mg per day and number of dosages thereof in the range of 4-6 doses per day with a gap of time in the range of 90-240 minutes between two dosages.

18. The method of claim 16, comprising providing the formulation in a unit dosage form in the range of 2000 mg-4000 mg per day.

19. The method of claim 16, comprising administering a number of dosages of the formulation in the range of 4-6 doses per day with a gap of time in the range of 90-240 minutes between two dosages.
